(19) United States
(12) Patent Application Publication
(10) Pub. No.: US 2009/0088405 A1
(43) Pub. Date: Apr. 2, 2009

(54) Personal lubricants with built-in or accompanying odorant delivery systems

(76) Inventors: Gary S. Kehoe, Phoenix, AZ (US); Stephen B. Roman, Phoenix, AZ (US)

Correspondence Address: TOD R NISSLE, PO BOX 55630, PHOENIX, AZ, 85078, US

(21) Appl. No.: 12/218,219

(22) Filed: Jul. 10, 2008

Related U.S. Application Data
(60) Provisional application No. 60/958,972, filed on Jul. 10, 2007.
(60) Provisional application No. 60/959,017, filed on Jul. 10, 2007.
(60) Provisional application No. 60/959,016, filed on Jul. 10, 2007.

(51) Int. Cl. A61K 8/73; A61Q 90/00

(52) U.S. Cl. 514/54

(57) ABSTRACT

A personal lubricant paired with an odorant release device.

This application claims priority based on provisional application 60/958,972, filed Jul. 10, 2007, provisional application 60/959,017, filed Jul. 10, 2007, and provisional application 60/959,016, filed Jul. 10, 2007.

I. The first disclosure herein relates to personal lubricants with built-in or accompanying odorant delivery systems to enhance sexual intimacy between partners prior to, during or after intercourse.

Personal lubricants that provide lubrication during sexual intercourse are known. Odorants that promote sexual intimacy are known. However, personal lubricants with built-in or accompanying odorant delivery systems to increase sexual intimacy are not known.

Several studies have shown how compounds that bind the pheromone receptor increase sexual intimacy between partners when in close proximity. Moreover, essential oils often used in aromatherapy and other odorants have also been shown to increase sexual intimacy between partners in close proximity. This invention provides a delivery system to add a pheromone(s), non-pheromone PR binder(s), and/or an odorant(s) to the atmosphere before, during or after sexual intercourse to increase sexual intimacy.

This first disclosure relates to personal lubricants in a 2-compartment delivery system or paired with an odorant delivery device for use before, during or after intercourse.

The resulting product will combine a personal lubricant with devices such as, but not limited to, passive odorant delivery systems, active odorant delivery systems, atomizers, pull-tab release systems, wicking devices, air freshening devices built into the cap, tear tapes, saturated substrates, and/or reservoirs filled with odorants with air exposure controls.

The resulting compositions decrease the wall surface frictional coefficients in the vagina, increase the intimacy between sexual partners, and increase the retention of moisture in the vagina.

The lubricant matrix may also be delivered by a single, multiple, or metered dose system where the amount of lubricant released is designed to match the volume necessary to properly coat the vaginal canal.

This disclosure relates to liquid compositions comprising a lubricant and an odorant delivery system that is built-in or accompanying the product. The composition is novel in that it increases the sexual intimacy between partners using the lubricant before, during and after sex.

In one embodiment, the disclosure relates to liquid compositions comprising a lubricant such as propylene glycol and an odorant known to increase sexual intimacy such as a pheromone receptor-binding compound like pyrroline isovalerianate and/or the odorant lavender oil.

In another embodiment, the disclosure relates to liquid compositions comprising a lubricant such as carrageenan and an odorant known to increase sexual intimacy such as a pheromone receptor-binding compound like pyrroline isovalerianate or lavender oil.

For these compositions, it is preferred to use the following ingredients in the ranges of:

Lubricant

| | | |
|---|---|---|
| Lubricant, such as carrageenan | 0.25% to 10.0% | (w/w) |
| Water | 1% to 95% | (w/w) |
| pH adjuster, such as lactic acid | 0.01% to 5% | (w/w) |
| Preservative, such as methylparaben/ | 0.01% to 0.2% | (w/w) |
| Propylparaben | | |

Odorant device

| | | |
|---|---|---|
| Pheromone(s), such as androstenol | 1% to 95% | (w/w) |
| Non-pheromone PR binders, such as | 1% to 95% | (w/w) |
| Pyrroline isovalerianate | | |
| Odorants, such as lavender oil | 1% to 95% | (w/w) |

In the embodiments of the disclosure, the following is applicable:
The lubricant may consist of one or more of, but not limited to
silicone, dimethicone, ethylmethylcellulose, methylcellulose, carrageenan, glycerine, polyhidritic glycerols, carboxymethylcellulose, guar gums, propylene glycol, alginates,
pheromones, 16-androstenes, androstenol, androstadienone, estratetraenol, androsterone, androstanol, pyrroline isovalerianate, butirrate, compounds that bind the pheromone receptor, androstanol derivatives,
lavender oil, clove oil, musk oil, rose oil, sandalwood oil, jasmine oil, cinnamon oil, nutmeg oil,
preservatives as needed or required by the CFR-21, methyl paraben, propyl paraben, sorbate, sodium hydroxymethylglycinate
citric acid, hydrogen peroxide, lactic acid and/or sodium hydroxide.

### EXAMPLE 1

The compositions of the present disclosure may be prepared by any conventional technique. In this example, there is produced a lubricant containing a pheromone, non-pheromone PR binder and an odorant using the following method:
1. Carrageenan is metered, or weighed into a mixing vessel;
2. The preservative, which in this example is methylparaben/propylparaben, is added to the mixture and blended to uniformity;
3. The pH adjuster, which in example 1 is citric acid, is added to the mixture in order to reach the target pH of 4.0;
4. Further additives, medicaments, ingredients, etc. may be added at this stage and blended to uniformity.
5. The pheromone, which in example one is androstenol, the non-pheromone PR binding compound, which in example 1 is pyrroline isovalerianate and the odorant, which in example 1 is lavender oil, are added to the odorant delivery device.
6. The device is sealed until use by the consumer.

The liquid composition was prepared by mixing the following ingredients at the prescribed percentages.

Lubricant

| | |
|---|---|
| Carrageenan | 2% |
| Water | 97% |
| Citric acid | 0.13% |
| Methyl/propylparaben | 0.05% |

Odorant device

| | |
|---|---|
| Androstenol | 10% |
| Pyrroline isovalerianate | 10% |
| Lavender oil | 40% |

The process can be carried out by a batch process, a continuous process or a combination of the two. The order of addition of the ingredients can also be varied to yield acceptable product depending upon the types of ingredients being blended together and their respective physical characteristics.

The resulting lubricant composition may be scented or unscented, colored or colorless, flavored or flavorless, water-based, and/or may include humectants. The vaginal formula is preferably pH balanced with a range between 3.2 and 10.6, preferably 4.0. The viscosity range is between 200-30,000 cps, preferably 5,500 cps.

The compounds are added to the odorant delivery device and it is sealed until used by the consumer.

The liquid matrix is applied inside the vagina in an amount preferred by the users prior to intercourse to efficiently coat the vaginal canal. The odorant is released into the air to increase sexual intimacy prior to, during and/or after intercourse.

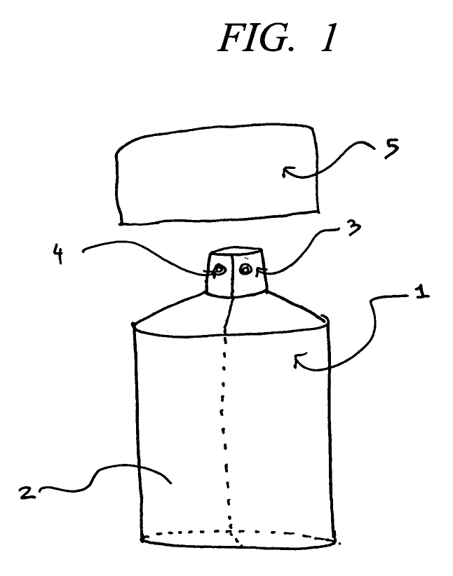

FIG. 1 illustrates a dispenser constructed in accordance with the invention.

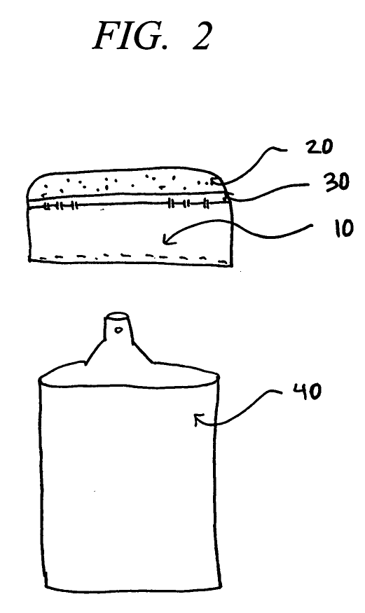

FIG. 2 illustrates an air freshener container.

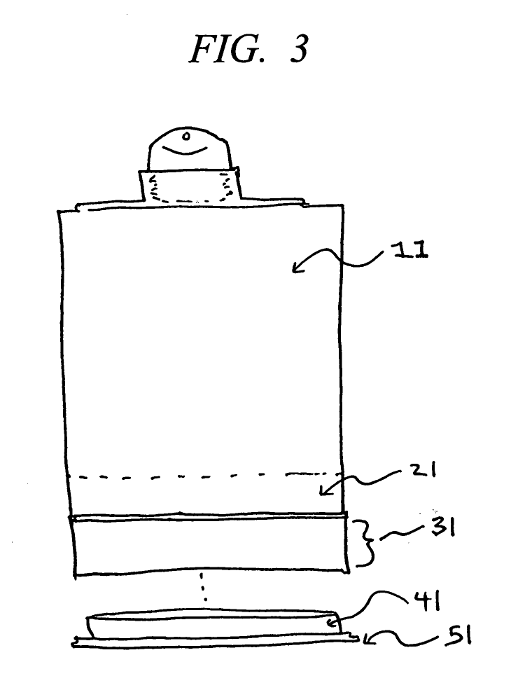

FIG. 3 illustrates an odorant releasing device.

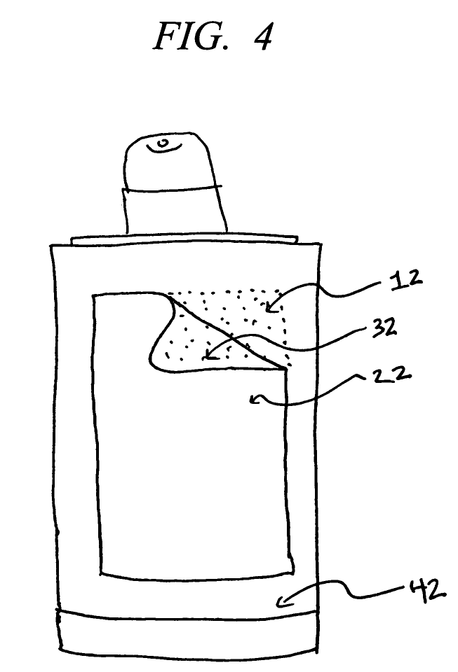

FIG. 4 illustrates odorant impregnated adhesive on a label.

The dispenser of FIG. 1 includes a first chamber, 1, a second chamber 2, a first dispensing nozzle 3, a second dispensing nozzle 4, and a cap 5.

The dispenser of FIG. 2 includes cap 10, odorant matrix 20, venting system 30, and bottle body 40.

The dispenser of FIG. 3 includes bottle body 11, piston 21, reservoir 31, odorant containing device 41, and bottle end cap 51.

The dispenser of FIG. 4 includes adhesive impregnated with odorant/fragrance 12, label front side 22, adhesive impregnated with odorant/fragrance 32, and bottle body 42.

II. A second disclosure herein relates to personal lubricants containing pheromones to increase intimacy between partners prior to, during and after intercourse.

Personal lubricants that provide lubrication during the act of sex are known. Pheromones that induce intimacy between partners are known. However, personal lubricants effectively containing pheromones are not known.

Pheromones have been studied and found to increase intimacy between partners when they are in close proximity. In the past, others have attempted to use pheromones in perfumes and colognes with little success due to the distance between the person wearing the pheromone and the other person who receives the scent. This invention adds the pheromone to a product, a personal lubricant, which is only used when a couple is about to engage in the act of sex. This makes for a favorable delivery device for the pheromone.

This second disclosure relates to liquid compositions comprising a lubricant which reduces the coefficient of friction, a pheromone, a pH adjuster, and a preservative resulting in compositions with viscosities and pH such that can be administered in the vaginal canal in humans. The composition may also incorporate a foaming agent. The proprietary composition is novel in that it incorporates pheromones to enhance intimacy between sexual partners.

The resulting liquid matrix/composition will be a mixture of, but not limited to: a lubricant, a pheromone, citric acid and a preservative such as methyl paraben and propyl paraben, Examples of lubricants that may be used are: silicone, dimethicone, ethylmethylcellulose, methylcellulose, carrageenan, glycerine, polyhidritic glycerols, carboxymethylcellulose, guar gums, propylene glycol. Other ingredients such as foam building agents, surfactants, medicaments, polyols, acids, bases, extracts, aromatics, charged ions, minerals, vitamins, and the like, may also be incorporated into the compositions.

The resulting compositions decrease the wall surface frictional coefficients, increase the intimacy between partners and increase retention of moisture in the vagina, In the case of foaming compositions, the foaming properties ensure an even distribution of the liquid along the vaginal epithelial layer. Specific formulations with combinations of various types of carrageenans, surfactants, and humectants produce foaming liquid compositions with properties allow for controlled retention of the liquid in the vaginal canal.

The foaming lubricant-pheromone matrix may also be delivered by a single, multiple, or metered-dose system that incorporates the correct amount of air into the liquid stream, thus controlling the level of resulting foam The metered pump makes it possible for the user to apply a specific/custom and repeatable amounts of the foaming composition from use to use. The foaming composition may also be used to deliver additional benefits/additives.

The amount of liquid dispensed will be designed to match that volume necessary to properly coat the vaginal canal.

This second disclosure relates to liquid compositions comprising a lubricant, a pheromone, an optional surfactant, an optional foam building agent, a pH adjuster and a preservative resulting in compositions with viscosities and pH such that can be administered in the vaginal canal in humans. The composition is novel in that it increases the intimacy of the two partners using the lubricant before, during and after sex.

The resulting liquid matrix/composition will be a mixture of, but not limited to: silicone, androstenol, and, methylparaben/propylparaben. Other ingredients such as medicaments, acids, bases, extracts, aromatics, charged ions, minerals, vitamins, and the like, may also be incorporated into the compositions.

In one embodiment, the disclosure relates to liquid compositions comprising water, a humectant such as glycerin, a surfactant such as cocamidopropyl betaine, a foam building agent such as xanthan gum, a lubricant such as propylene glycol a pheromone such as androstenol, a pH adjuster such as citric acid, and a preservative, such as methylparaben/propylparaben. Other ingredients such as medicaments, acids, bases, extracts, aromatics, charged ions, minerals, vitamins, and the like, may also be incorporated into the compositions.

In another embodiment, the disclosure relates to liquid compositions comprising water, a humectant such as glycerin, a surfactant such as cocamidopropyl betaine, a foam building agent such as xanthan gum, carrageenan such as iota and lambda carrageenan, a pheromone such as androstenol, a pH adjuster such as citric acid, and a preservative, such as methylparaben/propylparaben. Other ingredients such as medicaments, polyols, bases, extracts, aromatics, charged ions, minerals, vitamins, and the like, may also be incorporated into the compositions.

In another embodiment, the disclosure relates to liquid compositions comprising a lubricant such as propylene glycol, a pheromone such as androstenol, a pH adjuster such as citric acid, and a preservative, such as methylparaben/propylparaben, and a warming agent such as menthol. Other ingredients such as acids, bases, extracts, aromatics, charged ions, minerals, vitamins, and the like, may also be incorporated into the compositions.

In another embodiment, the disclosure relates to a liquid composition comprising 98.82% propylene glycol, 1.00% pheromone such as androstenol, 0.13% pH adjuster such as citric acid, and 0.05% preservative such as methlyparaben/propylparaben.

In producing the liquid compositions of the present disclosure, there are preferred amounts of lubricant, pheromone, pH adjuster, and preservative, when used. More particularly, it is preferred to use the following ingredients in the ranges of:

| | | |
|---|---|---|
| Lubricant, such as propylene glycol | 25% to 99.9% | (w/w) |
| Pheromone, such as androstenol | 0.001% to 10% | (w/w) |
| pH adjuster, such as citric acid | 0.01% to 2.5% | (w/w) |
| Preservative, such as methylparaben/ | 0.01% to 5% | (w/w) |
| propylparaben | | |

In producing the liquid compositions of the present disclosure, there are still preferred amounts of lubricant, pheromones, pH adjuster, and preservative, when used. More particularly, it is preferred to use the following ingredients in the ranges of:

| | | |
|---|---|---|
| Lubricant, such as androstenol | 85% to 99.9% | (w/w) |
| Pheromone, such as androstenol | 0.01 to 2% | (w/w) |
| pH adjuster, such as citric acid | 0.05% to 0.3% | (w/w) |
| Preservative, such as methylparaben/ | 0.01% to 0.2% | (w/w) |
| propylparaben | | |

In the embodiments of the disclosure, the following is applicable.
The lubricant may consist of one or more of, but not limited to, the following: silicone, dimethicone, ethylmethylcellulose, methylcellulose, carrageenan, glycerine, polyhidritic glycerols, carboxymethylcellulose, guar gums, or propylene glycol.

Androstenol (5a-16-androsten-3a-ol) or other pheromones may be used as desired.

Preservatives can be substituted for others as needed or required by the CFR-21.

Citric Acid and Sodium Hydroxide are used to adjust the pH as necessary to maintain the desired pH.

Any of the aforementioned ingredients may be used alone or in combination.

### EXAMPLE 1

The compositions of the present disclosure may be prepared by any conventional technique. In this example, there is produced a lubricant containing a pheromone liquid using the following method:
(a) Propylene Glycol is metered, or weighed into a mixing vessel;
(b) The preservative, which in Example 1 is methylparaben/propylparaben, is added to the mixture and mixed to uniformity;
(c) The pH adjuster, which in the Example 1 is citric acid, is added to the mixture in order to reach the target pH of 4.0
(d) The pheromone, which in Example 1 is androstenol, is added to the mixture and mixed to uniformity;
(e) The surfactant (when used), which in Example 1 is cocamidopropyl betaine, is added to the mixing vessel;
(f) The foam building agent (when used), which in Example 1 is xanthan gum, is added to the mixture. The agitation must not create a vortex, so as not to aerate the product. Aeration is not desired in the finished product;
(g) Further additives, medicaments, ingredients, etc. may be added at this stage and mixed to uniformity;
(h) The mixture is allowed to cool slightly and then it may be added to a foaming dispenser such as those supplied by Airspray or a normal airless dispenser such as those supplied by Megapumps.

The liquid composition was prepared by mixing the following ingredients at the prescribed percentages.

| | |
|---|---|
| Propylene Glycol | 99.5% (w/w) |
| Androstenol | 0.35% (w/w) |
| Citric acid | 0.13% (w/w) |
| Methylparaben/propylparaben | 0.05% (w/w) |

The process can be carried out by either a batch process, a continuous process or a combination of the two. The order of addition of the ingredients can also be varied to yield acceptable product depending upon the types of ingredients being blended to together and their respective physical characteristics.

The above example yields the preferred amount of pheromone and lubricant at a volume of 1.0 mL-5 mL (depending upon user preference).

The resulting pheromone lubricant composition may be scented or unscented, with color or colorless, flavored or flavorless, water-based, may include humectants like glycerin and surfactants as needed. The vaginal formula is preferably pH balanced with a range between 3.2 and 10.6, more preferably at 4.0. The viscosity range is between 200 and 30,000 cps, more preferably at 5,500 cps.

The liquid matrix is applied inside the vagina in an amount preferred by the users prior to intercourse to efficiently coat the vaginal canal. The preferred volume of liquid to apply in one application is 2.5 mL. This dosage most closely represents a volume necessary to simulate a natural volume of secretion in the vagina. Depending upon the type of container used, the pheromone matrix can be applied by depressing a nozzle that injects the foaming, liquid composition directly into the nose and/or throat. It may first be saturated onto a cotton swab that can be used to coat the walls of the vaginal canal. It may also be administered via an airless dispenser with a nozzle applicator.

III. A third disclosure herein relates to personal lubricants containing pheromones, nonpheromone pheromone receptor(PR)-binders, probiotics, prebiotics, vasodilators, and/or odorants to improve vaginal odor and increase sexual intimacy between partners prior to, during, and after sexual intercourse.

Personal lubricants that provide lubrication during sexual intercourse are known. Probiotic and/or prebiotic vaginal compositions that supplement the naturally-occurring vaginal microflora to improve urogenital health and odor are known. Pheromones and odorants that promote sexual intimacy are known. Vasodilators that enhance sexual pleasure and blood flow to the vaginal area are known, while the use of vasodilators combined with a stimulating inhalant, such as a nonpheromone pheromone receptor binder, pheromone, and/or odorant, with a personal lubricant is unknown. The use of lactic acid and hydrogen peroxide to reduce the growth of pathogenic bacteria in the vagina are known. However, a personal lubricant utilizing these compounds to improve vaginal odor and increase sexual intimacy and pleasure is not known.

Several studies have shown how pheromones and nonpheromone pheromone receptor binders increase sexual intimacy between partners when in close proximity. Studies have also shown that the species of bacteria colonizing the vagina play a large role in the intensity and pleasantness of vaginal odor. Offensive vaginal odor has been linked to species such as Streptococcus and Staphylococcus while more pleasant odors are linked to the Lactobacillus and Bifodobacterium species. Moreover, studies have shown that vaginal microflora play a major role in the effectiveness of pheromonal signals and attraction between sexual partners. Lactic acid is known to be produced by Lactobacillus bacteria and is effective in reducing the growth of pathogenic bacteria in the vagina as well as play a large role in maintaining a healthy pH in this area. Also, hydrogen peroxide is known to reduce the growth of pathogenic bacteria in the vagina. Vasodilators, such as arginine, nephridine, aminophylline, and peppermint oil, are known to increase blood flow and enhance sexual pleasure when applied to the vaginal area. In the past, others have attempted to use pheromones and nonpheromone pheromone receptor-binders in perfumes and colognes with little documented success. This is likely due to the distance between the wearer and the receiver and also the role of microfloral bacteria in the attractiveness of scent. Therefore, a personal lubricant is a favorable device for pheromones, nonpheromone pheromone receptor binders, probiotics, prebiotics, vasodilators, and/or odorants to increase sexual intimacy and pleasure between partners.

This third disclosure relates to liquid compositions comprising a lubricant that reduces the coefficient of friction, a member of group (a) or any combination thereof:
a) pheromone(s), nonpheromone PR-binder(s), probiotic(s), prebiotic(s), vasodilator(s), odorant(s),
   a pH adjuster, and a preservative resulting in compositions with viscosities and pH such that can be administered in the vaginal canal in humans. The proprietary composition is novel in that it incorporates the compounds named to improve vaginal odor and enhance sexual intimacy and pleasure between sexual partners.

The resulting liquid matrix/composition will be a mixture of, but not limited to: a lubricant, a member of group (a) or any combination thereof:
a) pheromone(s), nonpheromone PR-binder(s), probiotic(s), prebiotic(s), vasodilator(s), odorant(s),
   citric acid, and a preservative such as methyl paraben, propyl paraben, sorbate, or sodium hydroxymethylglycinate. Examples of lubricants that may be used are silicone, dimethicone, ethylmethylcellulose, methylcellulose, carrageenan, glycerine, polyhidritic glycerols, carboxymethylcellulose, guar gums, and/or propylene glycol. Because of the nature of probiotics, a separate delivery system may be used to keep these bacteria separate from the lubricant composition until use of the product. Other agents such as foam building agents, surfactants, medicaments, polyols, acids, bases, extracts, aromatics, charged ions, minerals, vitamins and the like may also be incorporated into the compositions.

The resulting compositions decrease the wall surface frictional coefficients, improve vaginal odor, increase intimacy and pleasure between sexual partners, and increase the retention of moisture in the vagina.

The lubricant matrix may also be delivered by a single, multiple, or metered dose system where the amount of lubricant released is designed to match the volume necessary to properly coat the vaginal canal.

This disclosure relates to liquid compositions comprising a lubricant, a member of group (a) or any combination thereof:
(a) pheromone(s), nonpheromone PR-binder(s), probiotic(s), prebiotic(s), vasodilator(s), odorant(s),
   a pH adjuster and a preservative resulting in compositions with viscosities and pH such that they can be administered in the vaginal canal in humans. The composition is novel in that it improves vaginal odor and increases the sexual intimacy and pleasure between partners before, during and after sex.

In one embodiment, the disclosure relates to liquid compositions comprising a lubricant such as propylene glycol, a member of group (a) or any combination thereof:
(a) pheromone(s) such as a compound classified as a 16-androstene, nonpheromone PR-binder(s) such as pyrroline isovalerianate, probiotic(s) such as any species of Lactobacillus, prebiotic(s) such as FOS, vasodilator(s) such as arginine, odorant(s) such as lavender oil,
   a pH adjuster such as citric acid, and a preservative, such as methyl- or propylparaben, and a warming agent such as menthol. Other ingredients such as medicaments, polyols, bases, extracts, aromatics, charged ions, minerals, vitamins, and the like may also be incorporated into the compositions.

In another embodiment, the disclosure relates to liquid compositions comprising a lubricant such as carrageenan, a member of group (a) or any combination thereof:
(b) pheromone(s) such as a compound classified as a 16-androstene, nonpheromone PR-binder(s) such as pyrroline isovalerianate, probiotic(s) such as any species of Lactobacillus, prebiotic(s) such as FOS, vasodilator(s) such as arginine, odorant(s) such as lavender oil,
   a pH adjuster such as citric acid, and a preservative, such as methylparaben/propylparaben, sorbate and/or sodium hydroxymethylglycinate.

In producing the liquid compositions of the present disclosure, there are preferred amounts of lubricant, pheromone, non-pheromone PR binding compound, odorant, pH adjuster, and preservative when used. More particularly, it is preferred to use the following ingredients in the ranges of:

| | | |
|---|---|---|
| Lubricant, such as carrageenan | 25% to 99.9% | (w/w) |
| Water | 1% to 95% | (w/w) |
| Pheromone(s), such as androstenol | 0.001% to 10% | (w/w) |
| nonpheromone PR-binder(s), such as | 0.001% to 1% | (w/w) |
| pyrroline isovalerianate | | |
| Probiotic(s), such as Lactobacillus | 0.001% to 1% | (w/w) |
| Prebiotics(s), such as FOS | 0.001% to 1% | (w/w) |
| Vasodilator(s), such as arginine | 0.001% to 1% | (w/w) |
| Odorant(s), such as lavender oil | 0.001% to 10% | (w/w) |
| pH adjuster, such as citric acid | 0.01% to 5% | (w/w) |
| Preservative, such as methylparaben/ | 0.01% to 0.2% | (w/w) |
| Propylparaben | | |

In the embodiments of the disclosure, the following is applicable:

The lubricant may consist of one or more of, but not limited to
silicone, dimethicone, ethylmethylcellulose, methylcellulose, carrageenan, glycerine, polyhidritic glycerols, carboxymethylcellulose, guar gums, propylene glycol, alginates,
pheromones, 16-androstenes, androstenol, androstadienone, estratetraenol, androsterone, androstanol, pyrroline isovalerianate, butirrate, compounds that bind the pheromone receptor, androstanol derivatives,
probiotics, Lactobacillus, Bifodobacteria, etc.
prebiotics, fructooligosaccharides, inulin sourced from chicory or agave,
vasodilators, arginine, nephridine, aminophylline, peppermint oil,
lavender oil, clove oil, musk oil, rose oil, sandalwood oil, jasmine oil, cinnamon oil, nutmeg oil,
preservatives as needed or required by the CFR-21,
citric acid, hydrogen peroxide, lactic acid and/or sodium hydroxide.

### EXAMPLE 1

The compositions of the present disclosure may be prepared by any conventional technique. In this example, there is produced a lubricant containing a pheromone, non-pheromone PR binder and an odorant using the following method:
1. Carrageenan is metered, or weighed into a mixing vessel;
2. The preservative, which in this example is methylparaben/propylparaben, is added to the mixture and blended to uniformity;
3. The pH adjuster, which in example 1 is citric acid, is added to the mixture in order to reach the target pH of 4.0;
4. The pheromone, which in example one is androstenol, is added to the mixture and blended to uniformity;
5. The nonpheromone pheromone receptor binder, which in example 1 is pyrroline isovalerianate, is added to the mixture and blended to uniformity;
6. The prebiotic, which in example 1 is FOS, is added to the mixture and blended to uniformity;
7. The vasodilator, which in example one is arginine, is added to the mixture and blended to uniformity;
8. The odorant, which in example 1 is lavender oil, is added to the mixture and blended to uniformity;
9. Further additives, medicaments, ingredients, etc. may be added at this stage and blended to uniformity.
10. The probiotic, which in example 1 is Lactobacillus, is blended with glycerine and added to the accompanying but compartment in the personal lubricant delivery device.

The liquid composition was prepared by mixing the following ingredients at the prescribed percentages.

| | |
|---|---|
| Carrageenan | 2% |
| Water | 97% |
| Androstenol | 0.35% |
| Pyrroline isovalerianate | 0.35% |
| Lavender oil | 0.10% |
| Arginine | 0.10% |
| Citric acid | 0.13% |
| Methyl/propylparaben | 0.05% |
| Lactobacillus | 0.1% |
| Glycerine | 5% |

The process can be carried out by a batch process, a continuous process or a combination of the two. The order of addition of the ingredients can also be varied to yield acceptable product depending upon the types of ingredients being blended together and their respective physical characteristics.

The resulting lubricant composition may be scented or unscented, colored or colorless, flavored or flavorless, water-based, and/or may include humectants. The vaginal formula is pH balanced with a range between 3.2 and 10.6, preferably 4.0. The viscosity range is between 200-30,000 cps, preferably 5,500 cps.

The liquid matrix is applied inside the vagina in an amount preferred by the users prior to intercourse to efficiently coat the vaginal canal.

IV. A fourth disclosure herein relates to personal lubricants containing pheromones, non-pheromone receptor binders and/or odorants to increase intimacy between partners prior to, during, and after intercourse.

Personal lubricants that provide lubrication during sexual intercourse are known. Pheromones, non-pheromone pheromone receptor (PR)-binding compounds and odorants that promote sexual intimacy are known. However, personal lubricants utilizing these compounds to increase sexual intimacy are not known.

Several studies have shown how pheromones increase sexual intimacy between partners when in close proximity. Similarly, non-pheromone PR binders have been shown to induce similar effects in people in close proximity. Moreover, essential oils often used in aromatherapy and other odorants have also been shown to increase sexual intimacy between partners in close proximity. In the past, others have attempted to use pheromones in perfumes and colognes with little documented success likely due to the distance between the pheromone-wearer and the receiver. This invention adds a pheromone(s), non-pheromone PR binder(s), and/or an odorant(s) to a personal lubricant, which is only used when a couple is about to engage in the act of sex. This makes for a favorable device for pheromones, non-pheromone molecules with similar activity, and odorants for this purpose because of the close proximity of the users.

The use of lactic acid and/or hydrogen peroxide in medical compositions to reduce growth of pathogenic bacteria is known. The use of these compounds as pH adjusters as well as to reduce the growth of pathogenic bacteria in a personal lubricant is unknown.

This fourth disclosure relates to liquid compositions comprising a lubricant that reduces the coefficient of friction, a member of group (a) or any combination thereof:

a) pheromone(s), non-pheromone PR binder(s), odorant(s),

a pH adjuster, and a preservative resulting in compositions with viscosities and pH such that can be administered in the vaginal canal in humans. The proprietary composition is novel in that it incorporates the compounds named to enhance sexual intimacy between sexual partners.

The resulting liquid matrix/composition will be a mixture of, but not limited to: a lubricant, a member of group (a) or any combination thereof:

b) pheromone(s), non-pheromone PR binders (s), odorant(s),

a pH adjuster, such as citric acid, hydrogen peroxide, and/or lactic acid, and a preservative such as methyl paraben, propyl paraben, sorbate, or sodium hydroxymethylglycinate. Examples of lubricants that may be used are silicone, dimethicone, ethylmethylcellulose, methylcellulose, carrageenan, glycerine, polyhidritic glycerols, carboxymethylcellulose, guar gums, and/or propylene glycol. Other agents such as foam building agents, surfactants, medicaments, polyols, acids, bases, extracts, aromatics, charged ions, minerals, vitamins and the like may also be incorporated into the compositions.

The resulting compositions decrease the wall surface frictional coefficients, increase the intimacy between sexual partners, and increase the retention of moisture in the vagina.

The lubricant matrix may also be delivered by a single, multiple, or metered dose system where the amount of lubricant released is designed to match the volume necessary to properly coat the vaginal canal.

This disclosure relates to liquid compositions comprising a lubricant, a member of group (a) or any combination thereof:

(b) pheromone(s), non-pheromone PR binders (s), odorant(s),

a pH adjuster and a preservative resulting in compositions with viscosities and pH such that they can be administered in the vaginal canal in humans. The composition is novel in that it increases the sexual intimacy between partners using the lubricant before, during and after sex.

In one embodiment, the disclosure relates to liquid compositions comprising a lubricant such as propylene glycol, a member of group (a) or any combination thereof:
(c) pheromone(s) such as a compound classified as a 16-androstene, non-pheromone PR binder(s) such as pyrroline isovalerianate, odorant(s) such as lavender oil,
   a pH adjuster such as citric acid, lactic acid and/or hydrogen peroxide, and a preservative, such as methyl- or propylparaben, and a warming agent such as menthol. Other ingredients such as medicaments, polyols, bases, extracts, aromatics, charged ions, minerals, vitamins, and the like may also be incorporated into the compositions.

In another embodiment, the disclosure relates to liquid compositions comprising a lubricant such as carrageenan, a member of group (a) or any combination thereof:
(a) pheromone(s) such as a compound classified as a 16-androstene, non-pheromone PR binder(s) such as pyrroline isovalerianate, and/or odorant(s) such as lavender oil,
   a pH adjuster such as lactic acid, and a preservative, such as methylparaben/propylparaben, sorbate and/or sodium hydroxymethylglycinate.

In producing the liquid compositions of the present disclosure, there are preferred amounts of lubricant, pheromone, non-pheromone PR binding compound, odorant, pH adjuster, and preservative when used. More particularly, it is preferred to use the following ingredients in the ranges of:

| | | |
|---|---|---|
| Lubricant, such as carrageenan | 0.25% to 10.0% | (w/w) |
| Pheromone(s), such as androstenol | 0.001% to 1% | (w/w) |
| Water | 1% to 95% | (w/w) |
| Non-pheromone PR binders, such as | 0.001% to 10% | (w/w) |
| Pyrroline isovalerianate | | |
| Odorants, such as lavender oil | 0.001% to 10% | (w/w) |
| pH adjuster, such as lactic acid | 0.01% to 5% | (w/w) |
| Preservative, such as methylparaben/ | 0.01% to 0.2% | (w/w) |
| Propylparaben | | |

In the embodiments of the disclosure, the following is applicable:

The lubricant may consist of one or more of, but not limited to
silicone, dimethicone, ethylmethylcellulose, methylcellulose, carrageenan, glycerine, polyhidritic glycerols, carboxymethylcellulose, guar gums, propylene glycol, alginates,
pheromones, 16-androstenes, androstenol, androstadienone, estratetraenol, androsterone, androstanol, pyrroline isovalerianate, butirrate, compounds that bind the pheromone receptor, androstanol derivatives,
lavender oil, clove oil, musk oil, rose oil, sandalwood oil, jasmine oil, cinnamon oil, nutmeg oil,
preservatives as needed or required by the CFR-21, methyl paraben, propyl paraben, sorbate, sodium hydroxymethylglycinate
citric acid, hydrogen peroxide, lactic acid and/or sodium hydroxide.

### EXAMPLE 1

The compositions of the present disclosure may be prepared by any conventional technique. In this example, there is produced a lubricant containing a pheromone, non-pheromone PR binder and an odorant using the following method:
1. Carrageenan is metered, or weighed into a mixing vessel;
2. The preservative, which in this example is methylparaben/propylparaben, is added to the mixture and blended to uniformity;
3. The pH adjuster, which in example 1 is citric acid, is added to the mixture in order to reach the target pH of 4.0;
4. The pheromone, which in example one is androstenol, is added to the mixture and blended to uniformity;
5. The non-pheromone PR binding compound, which in example 1 is pyrroline isovalerianate, is added to the mixture and blended to uniformity;
6. The odorant, which in example 1 is lavender oil, is added to the mixture and blended to uniformity;
7. Further additives, medicaments, ingredients, etc. may be added at this stage and blended to uniformity.

The liquid composition was prepared by mixing the following ingredients at the prescribed percentages.

| | |
|---|---|
| Carrageenan | 2% |
| Water | 97% |
| Androstenol | 0.35% |
| Pyrroline isovalerianate | 0.35% |
| Lavender oil | 0.10% |
| Citric acid | 0.13% |
| Methyl/propylparaben | 0.05% |

The process can be carried out by a batch process, a continuous process or a combination of the two. The order of addition of the ingredients can also be varied to yield acceptable product depending upon the types of ingredients being blended together and their respective physical characteristics.

The resulting lubricant composition may be scented or unscented, colored or colorless, flavored or flavorless, water-based, and/or may include humectants. The vaginal formula is preferably pH balanced with a range between 3.2 and 10.6, preferably 4.0. The viscosity range is between 200-30,000 cps, preferably 5,500 cps.

The liquid matrix is applied inside the vagina in an amount preferred by the users prior to intercourse to efficiently coat the vaginal canal.

This disclosure has been described with reference to various specific and illustrative embodiments and techniques. However, one skilled in the art will recognize that many variations and modifications may be made while remaining within the spirit and scope of the disclosure and claims.

We claim:

1. A personal lubricant paired with an odorant release device.
